(19) United States
(12) Patent Application Publication
(10) Pub. No.: US 2009/0126285 A1
(43) Pub. Date: May 21, 2009

(54) Facility Module for Production and Storage of Cell Therapy Product

(76) Inventors: Dong-Sam Suh, Seoul (KR); Chang-Kwon Ko, Seoul (KR); Seung-Ju Ryu, Gyeonggi-Do (KR); Sung-Jun Koh, Seoul (KR); Eun-Young Lee, Seoul (KR); Soo-Jin Jung, Seoul (KR); Dong-il Chang, Seoul (KR); Jun-Keun Lee, Gyeonggi-Do (KR); Hyun-Gi Yoon, Seoul (KR); Hyang-Soon Chang, Seoul (KR); Yong-Hyun Yoo, Seoul (KR); Jin-Wook Chung, Seoul (KR); Cheong-Ho Chang, Seoul (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/224,855

(22) PCT Filed: Mar. 16, 2006
(86) PCT No.: PCT/KR2006/000955

§ 371 Date: Sep. 8, 2008

(30) Foreign Application Priority Data
Mar. 10, 2006 (KR) 10-2006-0022827

(51) Int. Cl. E04H 5/00; C12M 3/00

(52) U.S. Cl. 52/79.1

(57) ABSTRACT

A facility module is provided for producing and storing a cell therapy product comprising: a Cell Therapy (CT) module one including separately prefabricated units having specific functions and separate entrances and exits so as to minimize contamination, and being capable of producing the cell therapy product, and a Banking of Cell and Tissue (BC) module Two including prefabricated units having specific functions and separate entrances and exits so as to minimize contamination, and being capable of appropriately storing hematopoietic stem cells, bone marrow cells and other cells for a prolonged period. It enables easy and low cost production of the cell therapy product, with sufficient quality to be transplanted into patients, within a short period, and permits clinical application to patients expeditiously. The present invention enables convenient installation and use of such a facility module anywhere adequate space is available, by providing the facility in a prefabricated module composed of specialized units according to function.

### BACKGROUND OF THE INVENTION

1. Field of the Invention

The present invention relates to a facility module for production and storage of a cell therapy product. More specifically, it is capable of easily producing a cell therapy product having a grade transplantable into patients within a short period of time and at a low production cost. It is also adapted to be clinically applicable to patients within an early time. It is provided in a prefabricated type composed of specialized units according to individual specific functions, and can be conveniently installed in any place where a predetermined amount of space is provided.

2. Related Prior Art

As is generally known, cell therapy technology, a next-generation technology which is expected to bring fundamental changes into the well-being trend peculiar to modern societies, and into the public health, pharmaceutical and medical industries supporting our aging society, is one of the most critical fields for technology-intensive and energy-saving advancement within the medical industry.

Cell therapy products are medicines used for the treatment, diagnosis and prevention of various diseases, produced by a series of necessary steps involving collecting and proliferating somatic cells from living bodies of patients themselves (autologous), other people (allogenic), or other animals (xenogenic), or differentiating stem cells into desired cell types, in order to repair impaired or defective cells or tissues and functions thereof. Such cell therapy products have a wide spectrum of applications, and over recent years, have been receiving a great deal of attention as a novel therapy having promising and unlimited potential for the treatment of various intractable diseases such as bums, cancers, senile dementia and others.

A lot of interest has been directed to cell therapy products as an important 21st-century new drug technology which is expected to be in the forefront of future life science and medical fields, as they have indefinite application fields depending upon the techniques being developed. Several hundred clinical tests and experiments on cell therapy products are being undertaken in technologically advanced countries including the USA, and a great deal of related research is also being actively undertaken in Korea. Diseases that can be treated by the use of cell therapy products may include, for example, various cancers, as well as intractable diseases such as hematologic/immunological disorders and diseases, neurological diseases, diabetes, bone/cartilage diseases and cardiovascular diseases. Further, conquest of intractable diseases via application of stem cells has become the crowning subject of the life science world in the 21st century, and as a result there have been technological innovations in all medical fields such as cardiovascular systems, nervous systems, blood and immune systems, genetic diseases, hepatic diseases, endocrinal diseases, bone, cartilage and skin diseases.

In recent years, the scientific world and many bio-venture companies have been actively conducting a great deal of research and study on cell therapy products, with some fruitful results, and therefore it is expected that the cell therapy products will be spotlighted as the medical industry aims at the world market. Experts in the related art propose that development of cell therapy products will make it possible to treat intractable diseases and substitute for organ transplants, and therefore will become a next-generation medical industry with an increasing market size.

As such, interest in and the necessity for cell therapy products, particularly autologous cell therapy products with established safety and effectiveness, have globally increased. However, considering the requirements that all factors, such as procedures and technologies for manufacturing the cell therapy product with quality acceptable for transplant into a patient, and that manufacturing facilities should be completely equipped, there is substantially no case in which such cell therapy products are provided by a single system. Therefore, the manufacture of cell therapy products and extension of the application of these products, is very difficult.

### SUMMARY OF THE INVENTION

Therefore, the present invention has been made in view of the above problems, and it is a first object of the present invention to provide a facility module for production and storage of a cell therapy product, comprising: a CT (Cell Therapy)-module capable of producing the cell therapy product, and a BC (Banking of Cell and Tissue)-module capable of storing hematopoietic stem cells and bone marrow cells and other cells, for a prolonged period of time, through appropriate processes.

For this purpose, a second object of the present invention is to provide a facility module for production and storage of a cell therapy product, wherein the CT and BC modules respectively comprise five functionally specialized units: a preparation unit, a processing unit, a microbial sterility test unit, a quality control unit and a utility unit.

A third object of the present invention is to enable production of a cell therapy product having a quality grade sufficient to transplant into patients, within a short period of time and at a low production cost, and enable clinical application thereof to patients in a timely manner, via use of the above-constituted facility module.

A fourth object of the present invention is to enable convenient installation and use of such a facility module in any place where a predetermined-size space is available, by providing the facility module in a prefabricated type composed of specialized units according to individual specific functions.

A fifth object of the present invention is to provide a facility module, for production and storage of a cell therapy product, which enables accomplishment of remarkably improved quality and reliability of the product and thereby enhances customer satisfaction.

In accordance with an aspect of the present invention, the above and other objects can be accomplished by the provision of a facility module for production of a cell therapy product, comprising a CT (Cell Therapy)-module composed of a plurality of separately prefabricated units having individual-specific functions, and having an entrance and exit separately partitioned from each other so as to minimize occurrence of contamination., The CT (Cell Therapy)-module includes a preparation unit (requiring wearing a clean room garment to enter sterile clean zones), for preparing/sterilizing raw materials and storing finished or semi-finished products; a processing unit (for maintaining cleanliness) to produce cell therapy products such as cultured chondrocytes and cultured osteoblasts; a microbial sterility test unit for examining the presence of microbial contamination (such as by bacteria) during the incubation period for production of cell therapy products; a quality control unit for confirming safety and effectiveness of the cell therapy products; and a utility unit for maintaining essential items such as a desired level of cleanliness, constant temperature and humidity, fire service and electric power for the other units.

In accordance with another aspect of the present invention, there is provided a facility module for storage of a cell therapy product, comprising a BC-module composed of a plurality of separately prefabricated units having specific functions and having an entrance and exit separately partitioned from each other so as to minimize occurrence of contamination, and being capable of storing hematopoietic stem cells and bone marrow cells and other cells for a prolonged period of time through appropriate processes. The BC module comprises: a preparation unit (Requiring wearing a clean room garment to enter sterile clean zones) for preparing/sterilizing raw materials; a processing unit for processing and storing the umbilical cord blood; a microbial sterility test unit for examining the presence of microbial contamination (such as by bacteria) during transportation or processing of the umbilical cord blood; a quality control unit for confirming safety and effectiveness of the cell therapy products; and a utility unit for maintenance of essential items such as a desired level of cleanliness, constant temperature and humidity, fire service and electric power for the other units.

### BRIEF DESCRIPTION OF THE DRAWINGS

FIG. 1 is a schematic plan block diagram of a cell therapy product CT (Cell Therapy) module applied to the present invention.

FIG. 2 is a front cross-sectional view of a preparation unit and a utility unit applied to the present invention.

FIG. 3 is a front cross-sectional view of a processing unit and a utility unit applied to the present invention.

FIGS. 4 through 7 are respectively top, front and left/right side views of a first air shower applied to the present invention.

FIGS. 8 through 10 are respectively plan, front and left/right side views of a second air shower applied to the present invention.

FIGS. 11 through 13 are respectively plan, front and side views of a pass box applied to the present invention.

FIGS. 14 through 16 are respectively plan, front and side views of a first HEPA (High Efficiency Particulate Air) filter unit applied to the present invention.

FIGS. 17 through 19 are respectively plan, front and side views of a second HEPA (High Efficiency Particulate Air) filter unit applied to the present invention.

FIGS. 20 through 22 are respectively plan, front and side views of an air handling part applied to the present invention.

FIG. 23 is a schematic plan block diagram of a cell therapy product BC (Banking of Cell and Tissue) module applied to the present invention.

FIG. 24 is a front cross-sectional view of a preparation unit and utility unit of FIG. 23.

### DETAILED DESCRIPTION OF THE PREFERRED EMBODIMENTS

The preferred embodiments of the present invention for accomplishing the above-mentioned objects will now be described in more detail with reference to the accompanying drawings.

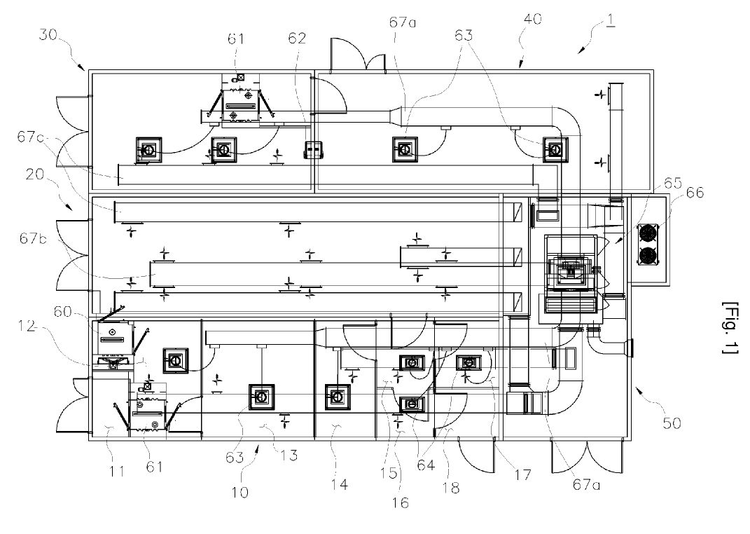

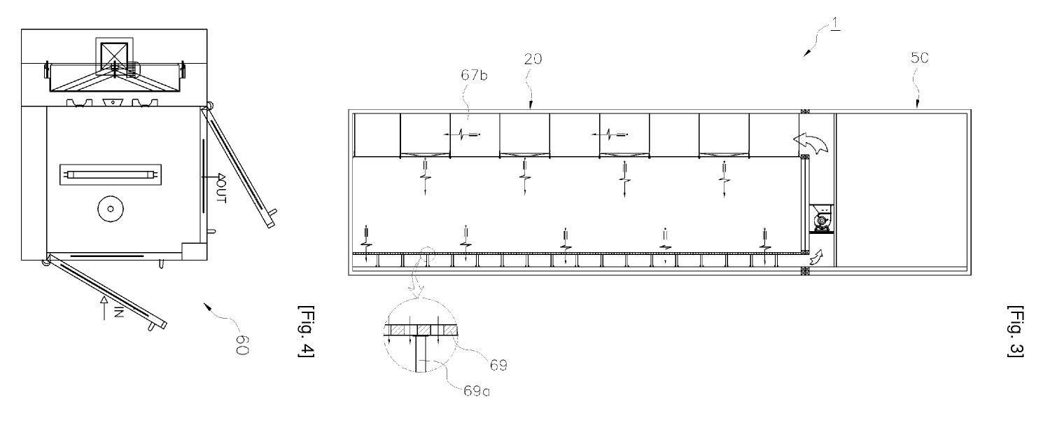

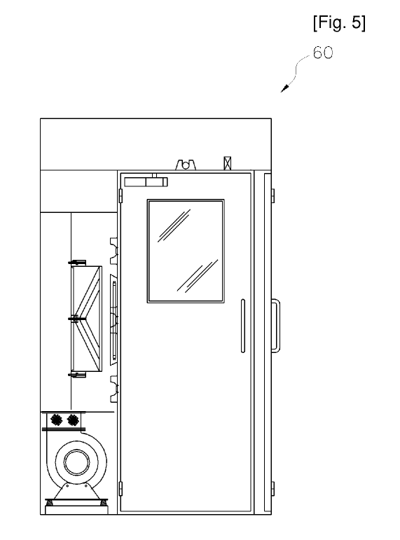

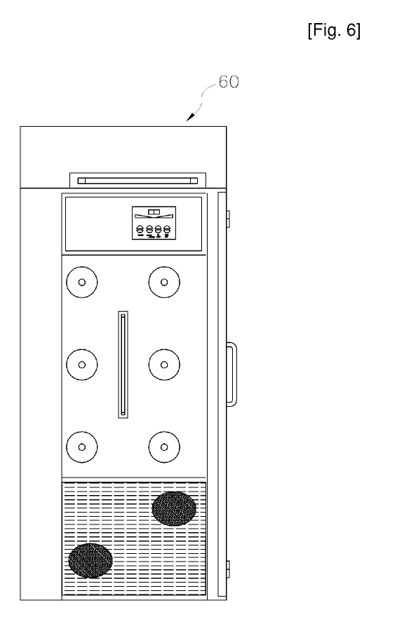

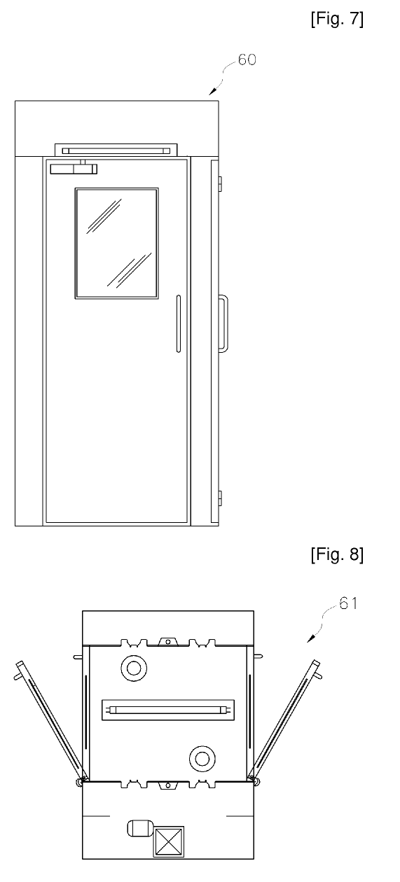

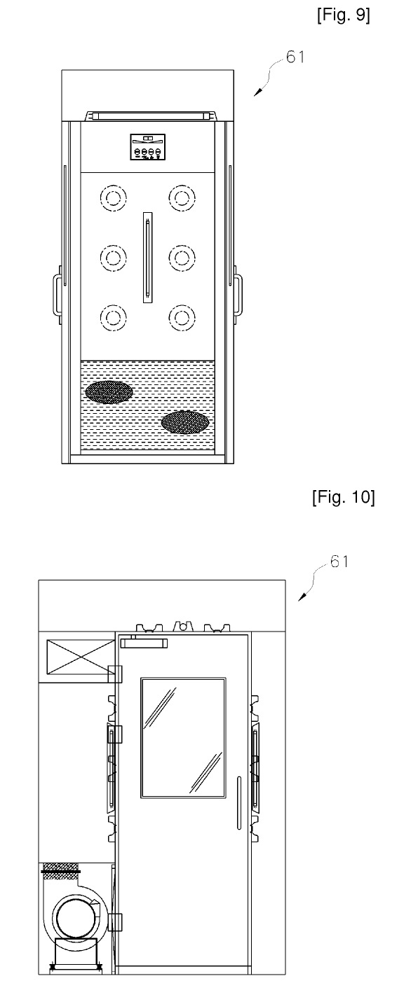

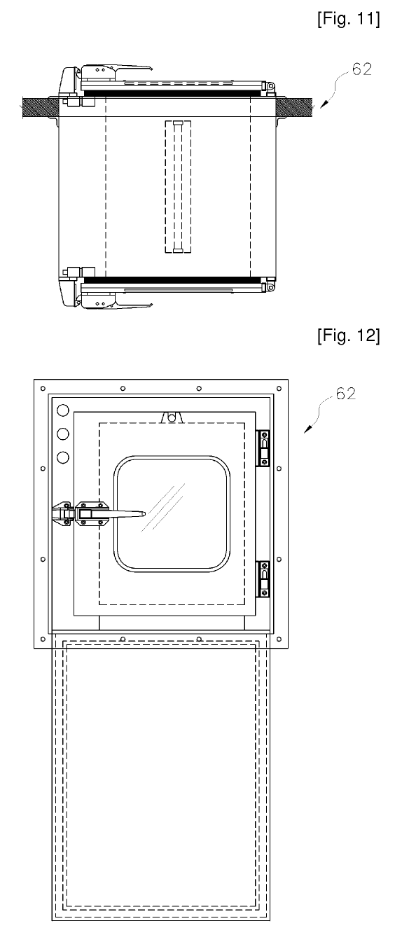

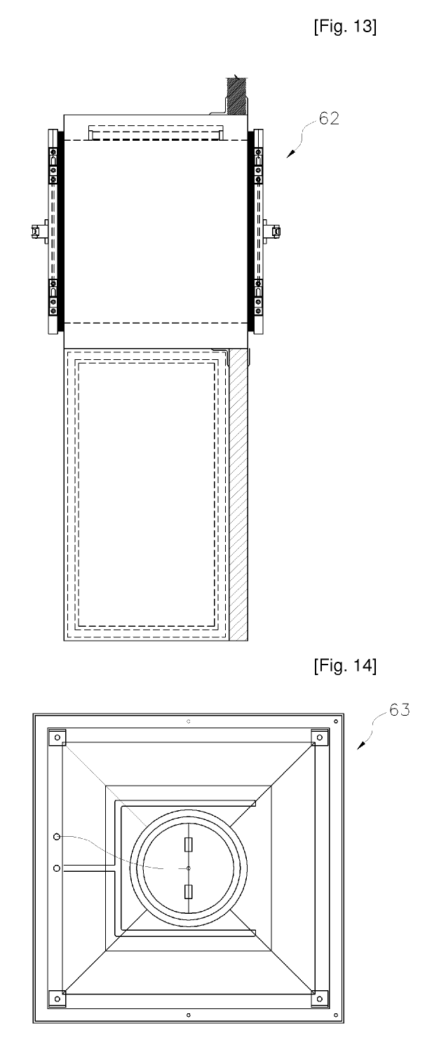

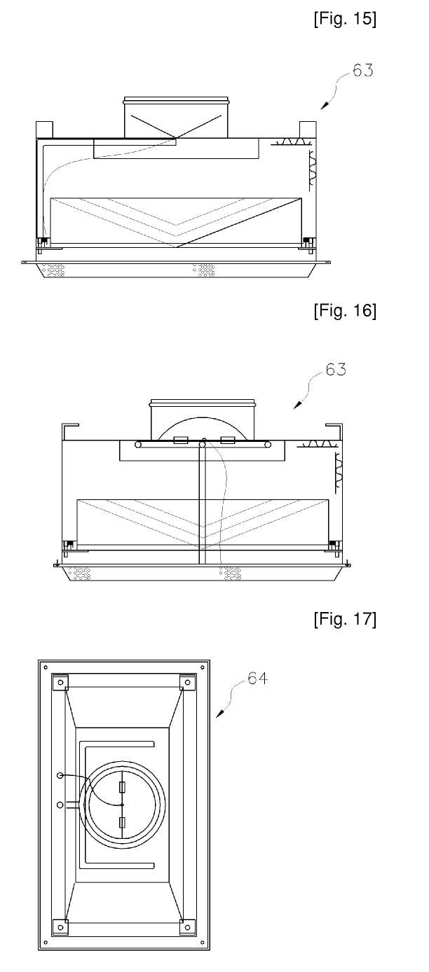

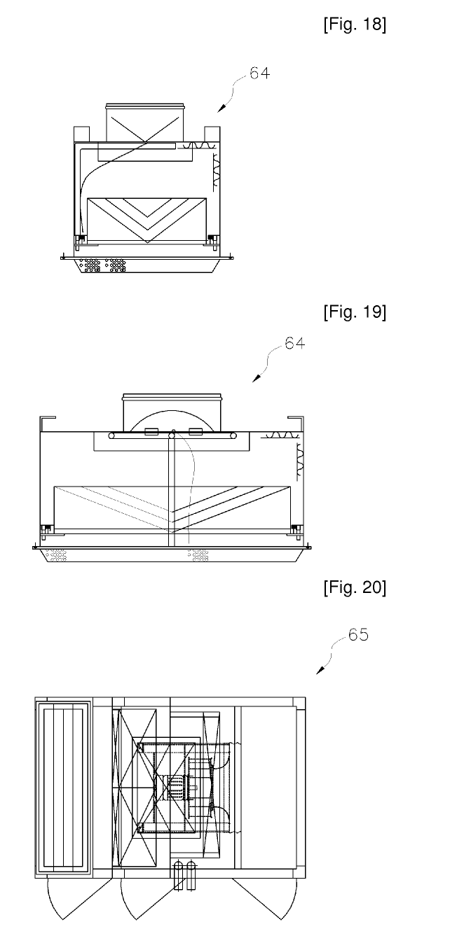

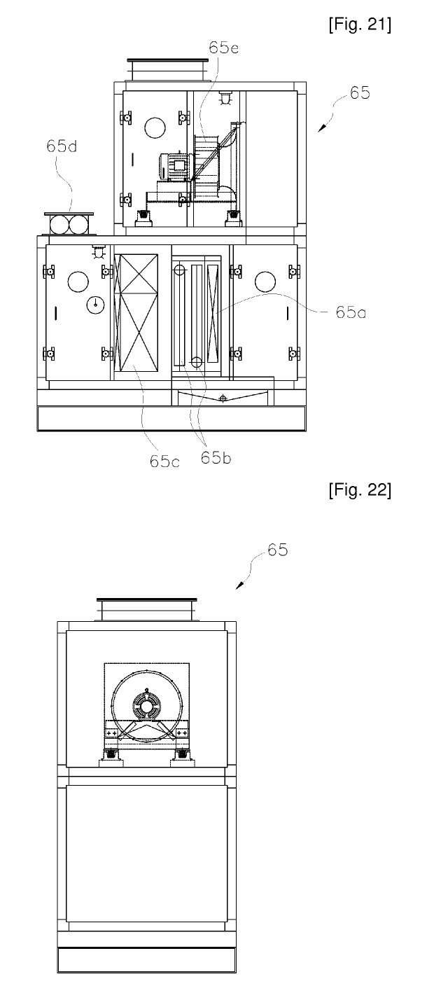

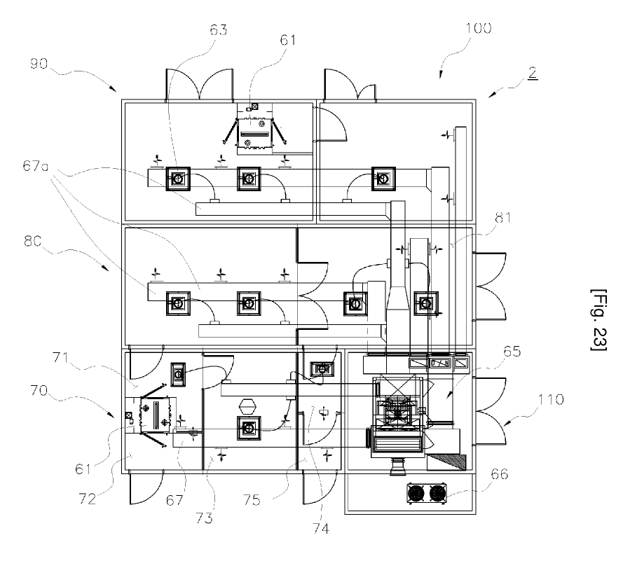

A facility module for production and storage of a cell therapy product, which is applied to the present invention, is constituted as shown in FIGS. 1 through 24.

In the description of the present invention which follows, if it is considered that description of known functions or constructions related to the present invention may make the subject matter of the present invention unclear, the detailed description thereof will be omitted.

Terms which will be described hereinafter are established taking into consideration functions in the present invention and may vary according to manufacturer's intention or general practices in the related art. Therefore, the terms used herein should be defined based on the contents of the specification of the present invention.

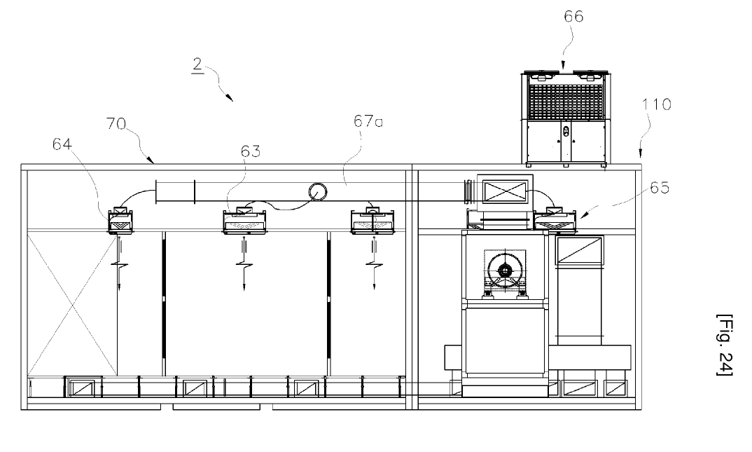

The present invention is directed to a facility module for production and storage of a cell therapy product, comprising: a CT (Cell Therapy)-module 1 (see FIG. 1) including a plurality of separately prefabricated units having individual-specific functions and having separately partitioned entrance and exit so as to minimize occurrence of contamination, and being capable of producing the cell therapy product; and a BC (Banking of Cell and Tissue)-module 2 (see FIG. 23) including a plurality of separately prefabricated units having individual-specific functions and having separately partitioned entrance and exit so as to minimize occurrence of contamination, and being capable of storing hematopoietic stem cells and bone marrow cells and other cells for a prolonged period of time through appropriate processes. Here, each module 1 and 2 is designed to follow a basic layout taking into account a minimal space necessary for processes and optimal size and weight advantageous for transportation.

Hereinafter, such technical constitution of the present invention will be described in more detail.

That is, as shown in FIG. 1, the CT (Cell Therapy)-module 1 is provided with a preparation unit 10, requiring wearing a clean room garment to enter sterile clean zones, for preparing/sterilizing raw materials and storing finished or semi-finished products.

In addition, the CT module 1 includes a processing unit 20 (for maintaining cleanliness) to produce cell therapy products such as cultured chondrocytes and cultured osteoblasts, at the rear of the preparation unit 10.

The facility module of the present invention also includes a microbial sterility test unit 30 for examining probable microbial contamination (such as by bacteria) during the incubation period for production of cell therapy products, at the rear of the processing unit 20.

At one side of the microbial sterility test unit 30, a quality control unit 40 for confirming safety and effectiveness of the cell therapy products is also provided.

Further, a utility unit 50 for maintenance of essential items such as a desired level of cleanliness, constant temperature and humidity, fire service and electric power for the respective units 10, 20, 30 and 40 is provided at one side of the preparation unit 10.

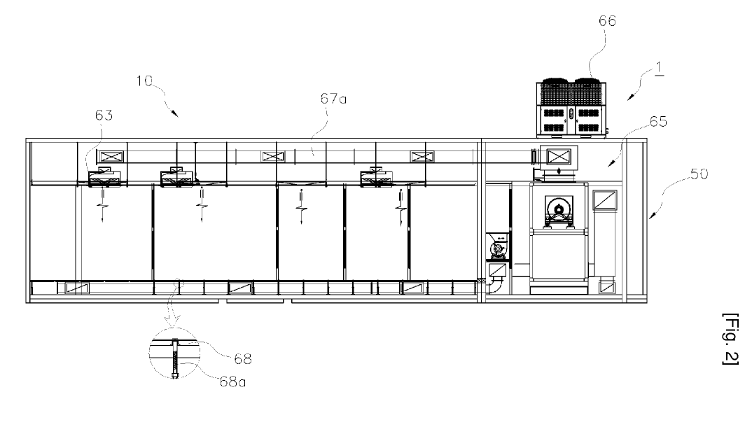

In accordance with the present invention, as shown in FIGS. 2 and 3, the preparation unit 10, processing unit 20, microbial sterility test unit 30 and quality control unit 40 (except utility unit 50) are permanently installed with sterile panels at a predetermined height from the bottom thereof. The preparation unit 10, microbial sterility test unit 30 and quality control unit 40 are provided with blank panels 68 at the top of multiple height-adjusting tools 68a arranged at regular intervals, and the processing unit 20 is provided with a grating panel 69 at the top of multiple supporting tools 69a arranged at regular intervals.

In addition, the module of the present invention includes an air handling part 65 provided inside the utility unit 50 and connected to an air cooler 66. The air handling part 65 is provided with an air filter 65a for preventing entrance of foreign materials, a cooling and heating coil 65b for heat exchange of fluid, a damper 65c for air volume control, a humidifier 65d for water level control, and a fan 65e for air volume control.

The air handling part 65 is connected with a first duct 67a, through which air is allowed to flow through the preparation unit 10, quality control unit 40 and microbial sterility test unit 30. The first duct 67a is provided with first HEPA (High Efficiency Particulate Air) filter units 63 provided at regular intervals, a second duct 67b discharging air to the inside of the processing unit 20, and a third duct 67c for flow of air into the respective units 10, 20, 30 and 40. In the third duct 67c, a plurality of second HEPA filter units 64 are provided at regular intervals.

Further, the inside of the preparation unit 10 is provided with a first dressing room 11 for wearing a first working uniform to enter a washing room or processing unit, a second dressing room 12 for wearing a clean room garment to enter the processing unit, a washing room 13 providing a space for washing, sterilizing and delivering articles to enter the processing unit and having an ultrapurification system, a packaging room 14 for packaging 5 products manufactured in the processing unit, a semi-finished product depository 17 for storing semi-finished products manufactured during processes in liquid nitrogen, a finished product depository 18 for final storage of finished products manufactured in the processing unit until shipment after packaging them in the packaging room 14, and first and second buffering zones 15 and 16 for providing clean conditions, serving as buffer areas with external the environment.

In addition, the facility module of the present invention further includes, as shown in FIGS. 1 and 4 through 10, first and second air showers 60 and 61 in the first dressing room 11 of the preparation unit 10, and further includes a second air shower 61 in the microbial sterility test unit 30, whereby entrance of contaminating particles from the outside is prevented upon entering clean zones and dust or bacteria adhered to the workers are washed and eliminated by high-velocity clean air.

Finally, in accordance with the present invention, between the microbial sterility test unit 30 and quality control unit 40 is a pass box 62 that enables only entrance and exit of articles without personnel entry, thereby preventing transfer of contamination source or clean air.

Meanwhile, although the preferred embodiments of the present invention have been disclosed with reference to the accompanying drawings, those skilled in the art will recognize that the present invention may be embodied in different forms with various modifications.

It should be understood that the drawings and detailed description thereof are not intended to limit the invention to the particular form disclosed, but on the contrary, the intention is to cover all modifications, equivalents and alternatives falling within the spirit and scope of the invention as defined by the appended claims.

Effects of the facility module for production of cell therapy product in accordance with the present invention, as constituted above, will be described hereinafter.

The CT-module 1 for production of the cell therapy product in accordance with the present invention comprises five units: the preparation unit 10; processing unit 20; microbial sterility test unit 30; quality control unit 40; and utility unit 50. The preparation unit 10 is composed of a dressing room for entering sterile clean zones, a washing room for preparing and washing raw materials/auxiliary materials used to manufacture products and a depository room for storing finished or semi-finished products of cell therapy products. The processing unit 20 is the place where cleanliness is kept at Class 100 levels and a variety of processes for isolating cells from tissues and differentiating/proliferating cells are carried out. The microbial sterility test unit 30 is a germ-free testing room where cleanliness is kept in Class 10000 levels and a sterility test is conducted on raw materials/auxiliary materials before and after processes and final products. The quality control unit 40 is the place where a variety of QC tests, except a sterility test, are conducted on raw materials or auxiliary materials before and after processing thereof, and on final products. The utility unit 50 is the place where equipment to constantly maintain temperature/humidity of the module and a desired level of cleanliness corresponding to the respective units is operated. Details thereof will be disclosed hereinafter.

In the facility module of the present invention, when an air handling part 65 is driven, air is circulated as indicated by arrows, through the respective ducts 67a, 67b and 67c and grating panel 69. Particularly, where the CT-module 1 is used, preparation of various media and reagents and sterilization of various implements and materials which are necessary for production of cell therapy products, is conducted in the preparation unit 10, and a variety of processes for isolating cells from tissues and differentiating/proliferating cells are conducted in the processing unit 20. For chondrocytes therapeutic, processes of producing the cell therapy products were carried out in the processing unit 20 of CT-module 1 as follows:

As a first step, cartilage isolation and primary culture were carried out as follows:

1) Biopsy specimen harvested from hospitals was transferred to the processing unit in the CT module, followed by isolation of cartilage.

2) The biopsy specimen was cut into small pieces on the sterile workbench, treated with enzymes and placed in a C02 incubator, followed by isolation of chondrocytes.

3) The chondrocytes thus isolated were cultured in a flask containing a culture medium, for about one month.

As a second step, media change and subculture were carried out as follows.

1) For one-month cell culture, chondrocytes were allowed to proliferate continuously.

2) Numbers of chondrocytes proliferated by about 500-fold from initial numbers of 1×10*5 cells to more than 5×10*7 cells immediately prior to manufacturing Cultured chondrocytes.

3) During proliferation of chondrocytes, media change was carried out to periodically supply nutrients to cells, and subculture was carried out to facilitate cell proliferation by changing a culture flask.

As a third step, a manufacturing process of chondrocyte therapeutic was carried out. For this purpose, test samples collected before and after processes and from final products were subjected to sterility tests in the microbial sterility test unit (30). Further, except for a sterility test, a variety of QC tests such as endotoxin test, mycoplasma test using PCR, cell count, cell viability test, virus test, cytotoxicity test and identity test were conducted in the quality control unit 40. Such processes for producing the cell therapy products were collectively carried out in the CT-module 1. After processes and QC tests for the products were complete, the chondrocyte therapeutic was transported to an operating room, followed by chondrocyte transplantation for the treatment of patients with cartilage defects.

The above-mentioned processes were carried out to manufacture chondrocyte therapeutic and bone cell therapy products. However, even though the CT-module 1 is capable of producing chondrocyte therapeutic and bone cell therapy products, such a module may also be used to produce other cell therapy products. When production technologies of chondrocyte therapeutic and bone cell therapy products are introduced in conjunction with the CT-module 1, it is possible to perform patient treatment using such cell therapy products and do business associated with treatment of patients.

Hereinafter, technical constitution of the BC (Banking of Cell and Tissue)-module 2 applied to the present invention will be described in more detail. In this description, details of technical constitution overlapping with those of the CT-module 1 will be omitted.

As shown in FIG. 23, the BC (Banking of Cell and Tissue)-module 2 is provided with a preparation unit 70 for wearing a clean room garment to enter sterile clean zones, and preparing/sterilizing raw materials. Here, the preparation unit 70 is provided with a first dressing room 72 for wearing a clean room garment to enter a washing room or processing unit; a washing room 73 providing a space for washing, sterilizing and delivering articles to enter the processing unit and including an ultrapurification system; first and second buffering zones 74 and 75 for providing clean conditions, serving as buffer areas with external environment; and a head room 71 serving as a buffer area to enter the processing unit.

In addition, a processing unit 80 for processing and storing the umbilical cord is provided at the rear of the preparation unit 70.

A microbial sterility test unit 90 for examining probable microbial contamination (such as by bacteria) during transportation or processing of the umbilical cord blood is also provided at the rear of the processing unit 80.

At one side of the microbial sterility test unit 90, a quality control unit 100 for confirming safety and effectiveness of the cell therapy products is also provided.

Further, at one side of the preparation unit 70, a utility unit 110 is provided for maintenance of essential items such as a desired level of cleanliness, constant temperature and humidity, fire service and electric power for the respective units 70, 80, 90 and 100.

In addition, the BC module of the present invention includes an air handling part 65 provided inside the utility unit 110 and connected to an air cooler 66; a first duct 67a connected to the air handling part 65 through the preparation unit 70, processing unit 80, quality control unit 100 and microbial sterility test unit 90; first and second HEPA filter units 63 and 64 connected at regular intervals to the first duct 67a; a third duct 67c for entry of air provided in the respective units 70, 80, 90 and 100; and second air showers 61 provided in the preparation unit 70 and microbial sterility test unit 90.

Effects of the facility module for storage of cell therapy product in accordance with the present invention, as constituted above, will be described hereinafter.

Similar to the CT-module for production of the cell therapy product, the BC-module 2 for storage of cell therapy product in accordance with the present invention also comprises five units: the preparation unit 70; processing unit 80; microbial sterility test unit 90; quality control unit 100; and utility unit 110. The preparation unit 70 is composed of a dressing room for entering sterile clean zones, and a washing room for preparing and washing raw materials or auxiliary materials necessary for manufacturing processes. The processing unit 80 is the place where cleanliness is kept in Class 10000 levels and a variety of processes for isolating cells from tissues or blood and storing cells are carried out. The microbial sterility test unit 90 is a germ-free testing room where cleanliness is kept in Class 10000 levels and a sterility test is conducted on raw materials or auxiliary materials before and after processing and cells for final storage. The quality control unit 100 is the place where a variety of QC tests except a sterility test are conducted on raw materials/auxiliary materials before/after processing thereof and cells for final storage. The utility unit 110 is the place where equipment necessary for constant maintenance of temperature/humidity of the module and cleanliness levels corresponding to the respective units is operated. Details thereof will be disclosed hereinafter.

Where the BC-module 2 of the present invention was used, preparation of various media and reagents and sterilization of various implements and materials, which are necessary for cell storage, were conducted in the preparation unit 70. A variety of processes for isolating cells from tissues or blood and storing cells were conducted in the processing unit 80. For storage of umbilical cord blood-derived hematopoietic stem cells, processing of storage cells were carried out in the processing unit 80 of BT-module 2 as follows.

As a first step, from the umbilical cord blood which was harvested from the umbilical cord, nucleated cells were isolated as follows.

1) A sample was collected from whole blood of the umbilical cord blood harvested from hospitals.

2) Nucleated cells were separated from the sample and were allowed to stand for separation of a red blood cell layer, followed by centrifugation to concentrate a nucleated cell layer.

3) After centrifugation was complete, the top plasma layer was removed using an Auto-Expressor, thereby leaving only a concentrate containing a small amount of the red blood cell layer and a concentrated layer of nucleated cells in the blood unit collection bag.

As a second step, a packaging step was carried out as follows.

1) The concentrated layer of nucleated cells separated in the first step was transferred to a freezing bag with removal of air contained therein.

2) The freezing bag containing the nucleated cell concentrates was placed in a case, followed by sealing.

3) Bar cord was attached to the freezing bag.

4) The freezing bag was packaged to prevent the risk of contamination and was finally inserted into a canister to prepare a finished product.

As a third step, freezing and storage processes were carried out as follows.

1) The finished canister was put into a frame and placed in a freezer equipped with an automatic thermostat.

2) A freezing program was operated to initiate freezing.

3) The thus-frozen sample was stored in a liquid nitrogen storage container.

4) Thereafter, in order to demonstrate safety and effectiveness of BabyCell, a quality control was carried out as follows.

For this purpose, test samples collected from raw materials or auxiliary materials before or after processing thereof and cells for final storage were subjected to sterility test in the microbial sterility test unit (90). Further, a variety of QC tests such as cell count, cell viability, hematopoietic stem cell count and colony-forming unit (CFU) assay were also conducted. The above-mentioned processes were carried out to separate and store hematopoietic stem cell from the umbilical cord blood. Therefore, even though the BC-module 2 is the facility capable of separating and storing umbilical cord blood-derived hematopoietic stem cells, such a module may also be used to process and store cell types other than hematopoietic stem cells. When technologies for separation and storage of hematopoietic stem cells from the umbilical cord blood are introduced in conjunction with the BC-module 2, it is possible to do business associated with separation and storage of hematopoietic stem cells.

As apparent from the foregoing, the present invention provides a facility module for production and storage of a cell therapy product, comprising: a CT (Cell Therapy)-module capable of producing a cell therapy product; and a BC (Banking of Cell and Tissue)-module capable of storing hematopoietic stem cells and bone marrow cells and other cells for a prolonged period of time through appropriate processes. The CT and BC modules are respectively composed of five functionally specialized units: a preparation unit; a processing unit; a microbial sterility test unit; a quality control unit; and a utility unit. Therefore, the present invention enables easy production of the cell therapy product having a quality grade sufficient to transplant into patients within a short period of time and at a low production cost. In addition, the present invention enables convenient installation and use of such a facility module in any place where a predetermined-size space is secured, by provision of the facility module in a prefabricated state composed of specialized units according to the individual functions. Consequently, the present invention enables accomplishment of remarkably improved quality and reliability of the product and thereby enhanced customer satisfaction.

What is claimed is:

1-10. (canceled)

11. A facility module for production of a cell therapy product, comprising:
a Cell Therapy (CT)-module (1) composed of a plurality of separately prefabricated units having individual-specific functions, having an entrance and exit separately partitioned from each other so as to minimize occurrence of contamination, and being capable of producing the cell therapy product,
the CT-module (1) including
a preparation unit (10) for wearing a clean room garment to enter sterile clean zones, preparing/sterilizing raw materials and storing finished/semi-finished products;
a processing unit (20) for maintaining a desired level of cleanliness to produce cell therapy products such as cultured chondrocytes and cultured osteoblasts;
a microbial sterility test unit (30) for examining for the presence of microbial contamination, such as by bacteria, during the incubation period for production of cell therapy products;
a quality control unit (40) for confirming safety and effectiveness of the cell therapy products; and
a utility unit (50) for maintaining essential items such as the desired level of cleanliness, constant temperature and humidity, fire service and electric power for the respective units (10, 20, 30, 40).

12. The facility module according to claim 11, wherein the preparation unit (10) processing unit (20), microbial sterility test unit (30) and quality control unit (40) have fixed panels installed at a predetermined height from the bottom, with the preparation unit, microbial sterility test unit and quality control unit provided with blank panels (68) at the top of multiple height-adjusting tools arranged at regular intervals, and the processing unit provided with a grating panel (69) at the top of multiple supporting tools arranged at regular intervals.

13. The facility module according to claim 11, wherein the module (1) further comprising:
an air handling part (65) provided inside the utility unit (50) and connected with an air cooler;
a first duct (67a) connected to the air handling part through the preparation unit, quality control unit and microbial sterility test unit;
first HEPA filter units (63) connected to the first duct at regular intervals;
a second duct (67b) connected to the air handling part and discharging air to the inside of the processing unit;
a third duct (67c) for entry of air provided in the respective units (10, 20, 30, 40), and
a plurality of second HEPA filter units (64) connected at regular intervals to the third duct.

14. The facility module according to claim 11, wherein the preparation unit (10) is further comprising that:
a first dressing room (11) for wearing a first working uniform to enter a washing room or processing unit;
a second dressing room (12) for wearing a clean room garment to enter the processing unit;
a washing room (13) providing a space for washing, sterilizing and delivering articles to enter the processing unit, and including an ultrapurification system;
a packaging room (14) for packaging products manufactured in the processing unit;
a semi-finished product depository (17) for storing, in liquid nitrogen, semi-finished products produced in the manufacturing processes;
a finished product depository (18) for final storage of finished products manufactured in the processing unit until packaging in the packaging room, and shipment; and
first and second buffering zones (15, 16) for providing clean conditions, serving as buffer areas with the external environment.

15. The facility module according to claim 14, wherein the first dressing room (11) of the preparation unit is further provided with first and second air showers (60, 61), and the microbial sterility test unit (30) is further provided with a second air shower (61), whereby entrance of contaminating particles from the outside is prevented from entering clean zones and dust or bacteria adhered to the workers are washed away and eliminated by high-velocity clean air.

16. The facility module according to claim 11, further comprising a pass box (62) that enables only entrance and exit of articles without personnel entry is provided between the microbial sterility test unit (30) and quality control unit (40), such that escape of a contamination source or clean air is prevented.

17. The facility module according to claim 13, wherein said air handling part (65) is provided with an air filter (65a), a cooling and heating coil (65b), a damper (65c), a humidifier (65d) and a fan (65e).

18. A facility module for storaging a cell therapy product, comprising
a Banking of Cell and Tissue (BC) module (2) composed of a plurality of separately prefabricated units having individual-specific functions and having an entrance and exit separately partitioned from each other so as to minimize occurrence of contamination, and being capable of storing hematopoietic stem cells and bone marrow cells and other cells for a prolonged period of time through appropriate processes,
wherein the BC module (2) includes:
a preparation unit (70) for wearing a clean room garment to enter sterile clean zones, and preparing/sterilizing raw materials;
a processing unit (80) for processing and storing the umbilical cord blood;
a microbial sterility test unit (90) for examining for the presence of microbial contamination, such as by bacteria, during transportation or processing of the umbilical cord blood;
a quality control unit (100) for confirming safety and effectiveness of the cell therapy products; and
a utility unit (110) for maintenance of essential items such as the desired level of cleanliness, constant temperature and humidity, fire service and electric power for the respective units (70, 80, 90, 100).

19. The facility module according to claim 18, wherein the preparation unit (70) is further comprising that:
a first dressing room (72) for wearing a clean room garment to enter a washing room or processing unit;
a washing room (73) providing a space for washing, sterilizing and delivering articles to the processing unit, and including an ultrapurification system;
first and second buffering zones (74, 75) for providing clean conditions, serving as buffer areas with external environment; and
a head room (71) serving as a buffer area to enter the processing unit.

20. The facility module according to claim 18, wherein the module further comprising that:
an air handling part (65) provided inside the utility unit (110) and connected with an air cooler;
a first duct (67a) connected to the air handling part through the preparation unit, processing unit, quality control unit and microbial sterility test unit;
first and second HEPA filter units (63, 64) connected at regular intervals to the first duct;
a third duct (67c) for entry of air provided in the respective units (70, 80, 90, 100); and
second air showers (61) provided in the preparation unit and microbial sterility test unit.
